(19) United States
(12) Patent Application Publication
(10) Pub. No.: US 2021/0106670 A1
(43) Pub. Date: Apr. 15, 2021

(54) TREATMENT AND PREVENTION OF GENITAL WARTS

(71) Applicant: Jack Silvers, Los Angeles, CA (US)

(72) Inventor: Jack Silvers, Los Angeles, CA (US)

(21) Appl. No.: 17/016,246

(22) Filed: Sep. 9, 2020

Related U.S. Application Data
(60) Provisional application No. 62/897,392, filed on Sep. 9, 2019.

(51) Int. Cl. A61K 39/12; A61K 33/16; A61P 31/20

(52) U.S. Cl. CPC A61K 39/12; A61K 38/14; A61P 31/20; A61K 33/16

(57) ABSTRACT

Described herein are methods of treating or preventing genital warts comprising administering to a subject a therapeutically effective amount of a vaccine against human papillomavirus (HPV) and reducing exposure of an area susceptible to genital warts to moisture, to non-neutrally electrically charged materials, or a combination thereof. Also described are kits or articles of manufacture for use in treating or preventing genital warts.

### FIELD OF THE INVENTION

Disclosed herein are methods of treating and preventing genital warts.

### BACKGROUND OF THE INVENTION

The human papillomavirus (HPV) family consists of over 170 different varieties of DNA virus, and most HPV infections cause no symptoms, and either remain dormant or resolve without the subject even realizing they were infected. But some individuals harboring HPV can be afflicted with lesions or other growths, and in some cases, such as HPV-mediated cervical cancer, symptoms do not manifest for years after exposure to the virus. Of the approximately 40 different HPV family members, a subset comprising HPV 6, 11, 42, and 44, is associated with condyloma accuminata (genital warts), a disease estimated to affect between 500,000 and a million individuals per year in the United States. HPV 6 and 11 account for over 90% of all genital wart cases.

Current treatment modalities for genital warts include topical treatments such as trichloroacetic acid and podophyllin, surgical excision or curettage of the wart(s), or cryotherapy. These methods can be invasive, inconvenient, and/or costly, and none ensure that the viral DNA residing in the infected cells will not manifest as additional warts subsequent to treatment. Further, administration of an HPV vaccine, e.g., GARDASIL® to humans who already have clinically visible condyloma accuminata has failed to affect their clearance.

Thus, there is a need for improved methods for treating and preventing genital warts associated with HPV infection. The disclosed methods are directed to these and other important needs.

### SUMMARY OF THE INVENTION

Disclosed herein are methods of preventing the onset of genital warts in a subject comprising administering to the subject a therapeutically effective amount of a vaccine for preventing human papilloma virus (HPV) infection and maintaining a dry genital region.

Also disclosed herein are methods of preventing recurrence of genital warts in a subject comprising administering a drying agent or absorbent composition to an area affected with genital warts.

Provided herein are methods of preventing the recurrence of genital warts in a subject comprising removing at least one genital wart from an affected area on the subject and applying a drying agent or absorbent composition to the affected area.

Methods are also provided for preventing genital warts in a subject comprising maintaining a dry genital region.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods, which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

As used herein, “affected area” refers to any area on the body where a genital wart is or was present on an individual. In most cases, affected areas will be the anogenital region of an individual or subject.

The term “subject” as used herein refers to any animal, but in particular humans. Thus, the methods are applicable to human and nonhuman animals, although preferably used with most preferably with humans. “Subject” and “patient” are used interchangeably herein.

As used herein, the phrase “therapeutically effective amount” refers to an amount of a vaccine, pharmaceutical, or topical treatment, as described herein, effective to achieve a particular biological or therapeutic result such as, but not limited to, biological or therapeutic results disclosed, described, or exemplified herein. The therapeutically effective dose may vary according to factors such as the state of HPV infection, age, sex, and weight of the individual, and the ability of the composition to cause a desired response in a subject. Such results include, but are not limited to, the treatment of genital warts, as determined by any means suitable in the art.

As used herein, “treating” and like terms refer to clearing or reducing clinically detectable lesions resulting from HPV infection; reducing the severity and/or frequency of genital warts; eliminating genital warts; reducing the likelihood of genital warts; and/or improving or remediating damage caused, directly or indirectly, by HPV infection and genital warts.

Various terms relating to aspects of the description are used throughout the specification and claims. Such terms are to be given their ordinary meaning in the art unless otherwise indicated. Other specifically defined terms are to be construed in a manner consistent with the definitions provided herein.

Some embodiments of the present disclosure provide methods for preventing genital warts on a subject comprising administering to the subject a therapeutically effective amount of a vaccine against HPV and reducing exposure of the genital region to moisture, non-neutrally electrically charged materials, or a combination thereof. In some aspects, the reducing exposure of the genital region to moisture comprises reducing exposure to sweat. For example, sweat moisture can be absorbed by some articles of clothing such as cotton undergarments. In some aspects, the neutrally charged articles of clothing or other wicking or absorbent materials are used to reduce moisture exposure. Materials such as polyester or rubber, having non-neutral charges, set the stage for the development of clinically visible HPV lesion.

Administering the vaccine may be accomplished by intravenous, intramuscular, intradermal, intraperitoneal, subcutaneous, by spray, by aerosol, mucosal, transdermal, by immersion, oral, intraocular, intratracheal, intranasal, pulmonary, or rectal delivery, or other means known to those skilled in the art. In some aspects, the administering comprises oral, epidermal, intradermal intramuscular, topical, intravenous, rectal, or sublingual administration. In some aspects, the vaccine against HPV is an anti-HPV 6 or 11 vaccine. In some aspects, the anti-HPV vaccine is an HPV vaccine effective against one or more of HPV types 6,11, 16, and 18. In some aspects, the anti-HPV vaccine is GARDASIL®.

Without being bound by theory, it is believed that HPV can incorporate within the DNA of epithelial cells, including the keratinocytes, and remain dormant. It is further believed that HPV is not a sexually transmitted disease, but it is a commensal of the epithelial cells, and when given the opportunity, it results in clinically visible lesions in a genetically predisposed person. Elimination of HPV containing epithelial cells and/or manifestation of a clinically detectable lesion depend on the interaction of HPV and the keratinocytes, and the immune cells with the keratinocytes. Environmental changes, such as increased moisture, friction, or direct or close contact with non-neutrally electrically charged materials may adversely affect the skin's barrier properties that promote innate protection against HPV. Exposure of the affected area to moisture, and in some instances friction, may affect the body's ability to interact defensively and normally with HPV and even reduce the effectiveness of other treatments. For example, subjects who receive repeated treatments with liquid nitrogen (cryotherapy) or podofilox (e.g., CONDYLOX®) without keeping the genital area dry are more susceptible to recurring genital warts compared to those patients who maintain a drier genital region.

As exposure to moisture may be a contributing factor in some HPV infections, this present disclosure contemplates moisture removal, avoidance, and/or prevention to maintain the barrier in its natural state of dryness as a means to prevent or reduce the occurrence of genital warts. Thus, in some aspects of the present disclosure reducing exposure of the genital region to moisture comprises employing at least one mechanical means for removing moisture from the region. In some embodiments, the moisture to which exposure is reduced is sweat moisture. A mechanical means for removing moisture can be, but is not limited to, a towel or other absorbent pad. The mechanical means for removing moisture can comprise a wicking material that removes moisture from the area likely to be affected by genital warts that either retains said moisture in the fabric or delivers it away from the area. Wicking undergarments, for example, can remove sweat from the genital area to maintain a relatively dry environment compared to wearing non-wicking undergarments.

Another mechanism for removing moisture includes applying at least one drying agent. For example, placing a desiccant in close proximity to the area of the body likely to be affected by genital warts can remove problematic moisture. Other drying agents can include pharmaceuticals, anti-perspirants, and iontophoresis devices that help prevent, eliminate, alleviate, reduce, or otherwise control excessive perspiration. In some aspects of the invention, exposure to moisture can be prevented, modified, eliminated, alleviated, or otherwise controlled by employing a mechanical means for removing moisture and applying a drying agent to remove moisture from the genital area to maintain the skin, including the stratum corneum, in its natural state.

In some embodiments of the present disclosure, preventing the onset of genital warts in a subject comprises administering to the subject a therapeutically effective amount of a vaccine against HPV and instructing the patient to reduce exposure of the genital area to moisture, non-neutrally electrically charged materials, or both. Physicians should inform patients that HPV can reside in the epidermal basal layer as a commensal and can even remain dormant within epidermal cells for a person's entire life after infection. Elimination of the HPV-harboring epithelial cells in a timely manner or hindering the development of a clinically detectable genital wart can be augmented by reducing exposure of potentially affected areas of the skin to moisture and to electrically non-neutral materials. Although individuals' tolerances of moisture exposure and/or electrically non-neutral material varies, the development of a detectable genital wart in a non-immunocompromised person is a manifestation of excessive exposure to moisture and/or electrically non-neutral material.

As patients will monitor their health status outside of a clinical setting, it is important that they are informed of the techniques described herein to lessen their likelihood of developing genital warts. Many subjects may believe that vaccination is completely effective at preventing genital warts, but individuals exposed to the virus prior to vaccination are still at risk of infection and developing these warts. The delayed onset of clinically detectable lesions may be responsible for an individual falsely assuming they are HPV-free at the time of vaccination. Further to this point, vaccination may also cause complacency, as the vaccinated individual may not be inclined to take any measures to lessen the probability of the onset of genital warts because they assume they are immune to HPV infection. Similarly, if a person's sexual partner is free of visible manifestations of HPV infection such as genital warts, the person may be more likely to engage in sexual contact, increasing the possibility of infection. Moreover, considering that HPV may be transmitted other than sexually, the absence of visible lesions may lead genetically predisposed persons to believe they are immune or non-infected. Without taking proper measures to prevent genital warts, medical intervention may be necessary once the person develops clinically visible verrucae.

The physician or other healthcare provider should provide instructions to the patient that exposure of the genital area to moisture should be reduced to maintain the skin in its natural state of dryness, even if the patient has been previously vaccinated against HPV. Explaining the proper way to dry the area with a mechanical means or to apply a drying agent as described above may accomplish this objective. The physician or other healthcare provider may also prescribe pharmaceuticals or recommend over-the-counter anti-perspiration treatments. The latter may be especially helpful for those afflicted with hyperhidrosis.

The physician or other healthcare provider should also explain the impact friction might have on a latent HPV infection in the areas associated with genital warts. Without being bound by theory, friction may be a substantial cofactor in the development of clinically detectable genital warts because it may increase the activation of latent HPV within the basal keratinocytes and/or increase the probability of HPV uptake by the epithelial cells. This activation and/or uptake may result in an increased rate of keratinocyte reproduction. The physician should also instruct the patient minimize exposure of the genital area to any type of material that may be, or become, electrically charged, such as synthetic materials, including polyester and rubber. At the very least, such contact with electrically charged materials should be reduced as much as possible, if not eliminated.

Some embodiments of the present disclosure describe methods of treating genital warts on a subject comprising removing at least one genital wart from the affected area on the subject and administering a drying agent or absorbent composition to the affected area. In some aspects of the embodiment, removing the at least one genital wart comprises laser removal therapy, liquid nitrogen therapy, or physical excision, and in some aspects removing the at least one genital wart comprises liquid nitrogen therapy. In some aspects, removing the at least one genital wart comprises administering a caustic agent including, for example cantharidin and CONDYLOX® (podofilox). In some aspects the caustic agent is cantharidin. In some aspects, removing the at least one genital wart comprises administering an acid. In some aspects, the acid is salicylic acid, or trichloroacetic acid. In some aspects, removing the at least one genital wart comprises administering a chemotherapeutic agent. In some aspects, the chemotherapeutic agent comprises 5-fluororuacil, bleomycin, or a combination thereof. In some aspects, removing the genital wart comprises administering an herbal extract. In some aspects, the herbal extract comprises sinecathechins.

The primary focus of wart treatment has traditionally been infection control measures through destructive modalities, excision, and, more recently, vaccination with synthetic proteins that are found on the capsule of HPVs. Restoration of the natural state of the barrier must be instructed to the patient first, and combined with the currently recommended treatment or preventative modalities to render them much more efficacious. Because asymptomatic individuals may be at risk of developing further warts, some embodiments presently disclosed are directed to methods of preventing recurrence of genital warts on an area of a subject previously affected with genital warts comprising administering a drying agent or absorbent composition to the area previously affected with genital warts. Especially in, but not limited to, instances of recurrence after treatment, a physician or healthcare provider may need to provide additional information to his/her patient to reduce the probability of recurrence. This disclosure contemplates the methods of preventing recurrence as described above as also further comprising instructing the subject to reduce exposure of moisture to the affected area. In addition to all of the above, or in place of instructing the patient to reduce exposure to moisture, some aspects of the embodiment further comprise administering a therapeutic amount of a pharmaceutical composition effective for treating at least one symptom associated with hyperhidrosis to the subject. Administration may be accomplished as described above.

Genital warts may resolve without treatment, while in some cases therapeutic intervention is either necessary or undertaken due to patient preference on the advice of a practicing physician. Unfortunately, the absence of a wart does not guarantee the absence of recurrence. Therefore, it is important for patients to take additional precautions to lessen the likelihood of recurrence, and one such additional precaution is to administer a drying agent, absorbent composition, or absorbent article of manufacture, to remove excess moisture from the genital region.

In some aspects of the present disclosure, a desiccant is used along with other treatment modalities to reduce exposure to moisture. For example, methods of preventing genital warts may be augmented by further comprising administering a desiccant to clothing that occludes the area. In some aspects, the occluding clothing is an undergarment, but it can be any clothing that can retain moisture. Occluding clothing can include tight fitting clothing. For this reason, reducing exposure to moisture, in some aspects of all of the methods presented herein, comprises wearing non-occlusive clothing. In some aspects, reducing exposure to moisture comprises reducing the wearing of occlusive clothing. In some aspects, reducing exposure to moisture comprises minimizing the wearing of occlusive clothing.

In some aspects, the present disclosure is directed to kits or articles of manufacture. The terms “kit” and “article of manufacture” are used synonymously herein. For use in the methods described herein, kits and articles of manufacture are also described. Such kits include a package or container that is compartmentalized to receive one or more therapeutically effective doses or amounts of a HPV vaccine disclosed herein. In one embodiment, the containers are formed from a variety of materials such as glass or plastic.

The articles of manufacture provided herein can contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, bags, containers, bottles, and any packaging material suitable for providing one or more therapeutically effective doses or amounts of a vaccine effective against HPV and intended mode of administration and treatment.

A kit typically includes labels listing contents and/or instructions for use, and package inserts with instructions for use. A set of instructions will also typically be included. Thus, the articles of manufacture provided herein can further contain a package insert that contains instructions for treating or preventing genital warts on an area of a subject susceptible to genital warts in accordance with the methods described herein, comprising instructions to reduce exposure of the area susceptible to genital warts to moisture, non-neutrally electrically charged materials, or a combination thereof. The articles of manufacture provided herein can optionally comprise a therapeutically effective amount of at least one desiccant or at least one mechanical means for removing moisture from the area susceptible to genital warts according to the present disclosure.

In one embodiment, a label is on or associated with the container. In one embodiment, a label is on a container when letters, numbers or other characters forming the label are attached, molded or etched into the container itself; a label is associated with a container when it is present within a package, container, receptacle, or carrier that also holds the container, e.g., as a package insert.

In one embodiment, a label is used to indicate that the contents are to be used for a specific therapeutic application. The label also indicates directions for use of the contents, such as in the methods described herein.

Those skilled in the art will appreciate that numerous changes and modifications can be made to the preferred embodiments of the invention and that such changes and modifications can be made without departing from the spirit of the invention. It is intended that the appended claims cover all such equivalent variations as fall within the true spirit and scope of the invention.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### EXAMPLES

The following examples are provided to further describe some of the embodiments disclosed herein. The examples are intended to illustrate, not to limit, the disclosed embodiments.

Thirty subjects (28 Caucasian and 2 Southeast Asian) presented with genital warts in the Los Angeles area. 28 of the subjects were male between 24 and 69 years old, with the majority (25 or 83%) being between 25 and 45 years old. Only 1 male was not circumcised. Three heterosexual male subjects exhibited perianal warts in addition to genital warts. Of these three subjects, one was diagnosed with hyperhidrosis, one admitted to obsessively cleaning himself after defecating, and one reported eating such quantities of very spicy food that he would experience burning in the perianal region. The two female subjects were 29 and 36 years old. Two subjects were taking prednisone for lupus erythematosus, and no other immunosuppressive factors were present in the cohort.

20 of the 30 subjects were diagnosed with hyperhidrosis, and 14 of the 30 complained of genital region moisture. Together, 21 of the 30 patients were either diagnosed with hyperhidrosis or complained of genital region moisture.

Thirteen subjects reported daily commutes of greater than 20 minutes. Seventeen subjects wore occlusive clothing (i.e., pants, undergarments) for greater than eight hours per day. 15 of these subjects wore denim for the majority of a calendar week. 28 of the 30 subjects either had genital region hyperhidrosis or engaged in behaviors such as wearing occlusive clothing and/or commuting for greater than 20 minutes per day.

### EMBODIMENTS

The following list of embodiments is intended to complement, rather than displace or supersede, the previous descriptions.

Embodiment 1. A method of preventing genital warts on an area of a subject susceptible to genital warts comprising: administering to the subject a therapeutically effective amount of a vaccine against human papillomavirus (HPV); and reducing exposure of the area susceptible to genital warts to moisture, non-neutrally electrically charged materials, or a combination thereof.

Embodiment 2. The method of embodiment 1, wherein the vaccine against HPV is an HPV 1, 2, 4, 57, 60, 63, 65, 66, or 156 vaccine, or any combination thereof.

Embodiment 3. The method of embodiment 1 or 2, wherein the administering comprises oral, epidermal, intramuscular, topical, intravenous, rectal, or sublingual administration.

Embodiment 4. The method of any one of embodiments 1 to 3, wherein reducing exposure of the genital area to moisture comprises employing at least one mechanical means for removing moisture from the genital area.

Embodiment 5. The method of any one of embodiments 1 to 3, wherein reducing exposure of the genital area to moisture comprises administering at least one drying agent.

Embodiment 6. The method of any one of embodiments 1 to 3, wherein reducing exposure of the genital area to moisture comprises employing at least one mechanical means and administering at least one drying agent to remove moisture from the genital area.

Embodiment 7. A method of preventing the onset of genital warts on a patient comprising: administering to the patient a therapeutically effective amount of a vaccine against human papilloma virus (HPV); and instructing the patient to reduce exposure of the genital area to moisture, non-neutrally electrically charged materials, or both.

Embodiment 8. The method of any one of embodiments 1 to 7, wherein the vaccine against HPV is effective against one or more of HPV types 6, 11, 16 and 18, e.g., GARDASIL®.

Embodiment 9. The method of embodiment 7 or 8, wherein the administering comprises oral, epidermal, intramuscular, topical, intravenous, rectal, or sublingual administration.

Embodiment 10. The method of any one of embodiments 7 to 9, wherein instructing the patient to reduce exposure of the genital area to moisture comprises instructing the patient to employ at least one mechanical means for removing moisture from the area.

Embodiment 11. The method of any one of embodiments 7 to 10, wherein instructing the patient to reduce exposure of the genital area to moisture comprises instructing the patient to administer at least one drying agent.

Embodiment 12. The method of any one of embodiments 7 to 11, wherein instructing the patient to reduce exposure of the genital area to moisture comprises instructing the patient to employ at least one mechanical means and administering at least one drying agent to remove moisture from the genital area.

Embodiment 13. A method of preventing recurrence of genital warts on a subject previously affected with genital warts comprising administering a drying agent or absorbent composition to the previously affected genital area.

Embodiment 14. A method of treating genital warts on an affected area of a subject comprising: removing at least one genital wart from the affected area on the subject; and administering a drying agent or absorbent composition to the affected area.

Embodiment 15. The method of embodiment 14, wherein removing the at least one genital wart comprises administering laser removal therapy, liquid nitrogen therapy, at least one caustic agent, at least one acid, at least one herbal extract, at least one chemotherapeutic agent, or physical excision, or a combination thereof.

Embodiment 16. The method of embodiment 15, wherein removing the at least one genital wart comprises administering liquid nitrogen therapy.

Embodiment 17. The method of embodiment 14, wherein removing the at least one genital wart comprises administering a caustic agent.

Embodiment 18. The method of embodiment 17, wherein the caustic agent is cantharidin. In other embodiments, the caustic agent is podofilox, e.g., CONDYLOX®. In other embodiments, the caustic agent comprises one or both of cantharidin or podofilox.

Embodiment 19. The method of embodiment 14, wherein the removing the at least one genital wart comprises administering at least one acid.

Embodiment 20. The method of embodiment 19, wherein the at least one acid is salicylic acid, trichloroacetic acid, or a combination thereof.

Embodiment 21. The method of embodiment 14, wherein removing the at least one genital wart comprises administering at least one herbal extract.

Embodiment 22. The method of embodiment 21, wherein the at least one herbal extract comprises sinecathecins. In other embodiments, the at least one herbal extract comprises ingenol mebutate, e.g., PICATO® gel. In other embodiments, the at least one herbal extract comprises both sinecathecins and ingenol mebutate.

Embodiment 23. The method of embodiment 14, wherein removing the at least one genital wart comprises administering a chemotherapeutic agent.

Embodiment 24. The method of embodiment 19, wherein the chemotherapeutic agent is 5-fluorouracil or bleomycin.

Embodiment 25. The method of any one of embodiments 14 to 22, further comprising instructing the subject to reduce exposure of moisture to the affected area.

Embodiment 26. The method of any one of embodiments 14 to 23 further comprising administering to the subject a therapeutic amount of a pharmaceutical composition effective for relieving at least one symptom associated with hyperhidrosis.

Embodiment 27. The method of embodiment 24, wherein administering a pharmaceutical composition effective for relieving symptoms associated with hyperhidrosis comprises oral, epidermal, intramuscular, topical, intravenous, rectal, or sublingual administration.

Embodiment 28. A method of preventing genital warts on a subject warts comprising reducing exposure of the genital area to moisture.

Embodiment 29. The method of embodiment 28, wherein the moisture is sweat moisture.

Embodiment 30. The method of embodiment 28, wherein reducing exposure of the genital area to moisture comprises administering a therapeutically effective amount of a pharmaceutical composition effective for relieving at least one symptom of hyperhidrosis.

Embodiment 31. The method of embodiment 30, administering a pharmaceutical composition effective for relieving at least one symptom associated with hyperhidrosis comprises oral, epidermal, intramuscular, topical, intravenous, rectal, or sublingual administration.

Embodiment 32. The method of any one of embodiments 28 to 31, further comprising administering a desiccant to any clothing or accessory that occludes the genital area.

Embodiment 33. The method of embodiment 32, wherein the occluding clothing or accessory is an undergarment.

Embodiment 34. The method of any one of embodiments 13 to 33 further comprising administering a therapeutically effective amount of a vaccine effective against human papilloma virus.

Embodiment 35. The method of embodiment 34, wherein the vaccine effect against human papillomavirus is effective against one or more of HPV types 6, 11, 16, and 18, e.g., GARDASIL®.

Embodiment 36. The method of any one of embodiments 1 to 12 or 23 to 35, wherein reducing moisture comprises minimizing wearing clothing comprising non-electrically-neutral material to a few hours per day or even less in the case of genital hyperhidrosis.

Embodiment 37 A kit comprising: a) a therapeutically effective amount of a vaccine effective against human papilloma virus; b) a package insert that contains instructions for treating or preventing genital warts in a subject susceptible comprising instructions to reduce exposure of the area susceptible to genital warts to moisture, non-neutrally electrically charged materials, or a combination thereof; and c) optionally, a therapeutically effective amount of at least one desiccant or at least one mechanical means for removing moisture from the area susceptible to genital warts.

1. A method of preventing the onset of genital warts in a subject comprising: administering to the subject a therapeutically effective amount of a vaccine against a human papilloma virus (HPV); and one or both of following:
i) d reducing exposure of the genital area to moisture, non-neutrally electrically charge materials, or a combination thereof; or
ii) instructing the subject to reduce exposure of the genital area to moisture, non-neutrally electrically charged materials.

2. The method of claim 1, wherein the vaccine against HPV is effective against one or more of HPV types 6, 11, 16, and 18.

3. The method of claim 1, wherein the administering comprises oral, epidermal, intramuscular, topical, intravenous, rectal, or sublingual administration.

4. The method of any one of claim 1, wherein reducing exposure of the area susceptible to plantar waits to moisture comprises employing at least on mechanical means for removing moisture from the area susceptible to plantar warts.

5. The method of any one of claim 1, wherein maintain a dry genital region comprises applying at least one drying agent.

6. The method of any one of claim 1, wherein reducing exposure of the genital region to moisture comprises employing at least on mechanical means and applying at least one drying agent to remove moisture from the genital region.

7. (canceled)

8. (canceled)

9. (canceled)

10. (canceled)

11. (canceled)

12. (canceled)

13. (canceled)

14. A method of preventing the recurrence of genital warts in a subject comprising:
a. removing at least one genital wart from an affected area on the subject; and
b. administering a drying agent or absorbent composition to the affected area.

15. The method of claim 14, wherein removing the at least one genital wart comprises administering laser removal therapy, liquid nitrogen therapy, at least one caustic agent, at least one acid, at least one herbal extract, at least one chemotherapeutic agent, or physical excision, or a combination thereof.

16. The method of claim 15, wherein removing the at least one genital wart comprises administering liquid nitrogen therapy.

17. The method of claim 14, wherein removing the at least one genital wart comprises administering a caustic agent.

18. The method of claim 17, wherein the caustic agent is cantharidin or podofilox.

19. The method of claim 14, wherein the removing the at least one genital wart comprises administering at least one acid.

20. The method of claim 19, wherein the at least one acid is salicylic acid, trichloroacetic acid, or a combination thereof.

21. The method of claim 14, wherein removing the at least one genital wart comprises administering at least one herbal extract.

22. The method of claim 21, wherein the at least, one herbal extract comprises sinecathecins or ingenol mebutate.

23. The method of claim 14, wherein removing the at least one genital wart comprises administering a chemotherapeutic agent.

24. The method of claim 23, wherein the chemotherapeutic agent is 5-fluorouracil or bleomycin.

25. The method of any one of claim 14, further comprising instructing the subject to reduce exposure of moisture to the affected area.

26. The method of any one of claim 14 further comprising administering to the subject a therapeutic amount of a pharmaceutical composition effective for relieving at least one symptom associated with hyperhidrosis.

27. (canceled)

28. (canceled)

29. (canceled)

30. (canceled)

31. (canceled)

32. (canceled)

33. (canceled)

34. (canceled)

35. (canceled)

36. (canceled)

37. A kit comprising:
a. a therapeutically effective amount of a vaccine effective against human papilloma virus;
b. a package insert that contains instructions for treating or preventing genital warts on an area of a subject susceptible to genital warts comprising instructions to reduce exposure of the area susceptible to genital warts to moisture, non-neutrally electrically charged materials, or a combination thereof; and
c. optionally, a therapeutically effective amount of at least one desiccant or at least one mechanical means for removing moisture from the area susceptible to genital warts.
